(19) United States
(12) Patent Application Publication
(10) Pub. No.: US 2022/0341061 A1
(43) Pub. Date: Oct. 27, 2022

(54) Wash-resistant bioactive cellulose fibre having antibacterial and antiviral properties

(71) Applicant: SMARTPOLYMER GmbH, Rudolstadt (DE)

(72) Inventors: Frank WENDLER, Rudolstadt (DE); Thomas SCHULZE, Rudolstadt (DE); Janine BAUER, Rudolstadt (DE)

(73) Assignee: SMARTPOLYMER GmbH, Rudolstadt (DE)

(21) Appl. No.: 17/725,719

(22) Filed: Apr. 21, 2022

(30) Foreign Application Priority Data
Apr. 21, 2021 (DE) 10 2021 110 053.4

(51) Int. Cl. D01F 1/10; B82Y 30/00; D01F 2/02; D06M 11/42; D06M 11/44

(52) U.S. Cl. CPC D01F 1/103; B82Y 30/00; D01F 2/02; D06M 11/42; D06M 11/44; D21H 11/20

(57) ABSTRACT

The invention relates to a cellulosic fibre loaded with a biologically active substance formed by the steps of: a) producing a cellulosic fibre loaded with ion exchanger, b) after-treating the fibre thus produced with an aqueous solution of a metal salt which exhibits antibacterial activity and/or antiviral activity, and c) after-treating the loaded fibre with an aqueous fixing solution to convert the metal salt into a water-insoluble form. The cellulosic fibres thus produced can be used to form textile fabrics, wound dressings, sanitary products, specialty papers, packaging or filter materials.

### CROSS-REFERENCE TO RELATED APPLICATIONS

This application claims priority to German Patent Application 10 2021 110 053.4 filed Apr. 21, 2021, which is hereby incorporated herein by reference in its entirety.

### FIELD OF THE INVENTION

The invention relates to a method for producing cellulosic shaped articles having long-lasting, wash-resistant antibacterial and antiviral properties, The method gives rise to uses in medicine (lab coats), hygiene (face mask), clothing (sport and leisure sector) and bedding (mattresses).

### BACKGROUND OF THE INVENTION

It is known that heavy metal ions, for example bismuth, silver, mercury, copper, zinc, tin or zirconium ions, act against pathogens, such as algae, bacteria, parasites, fungi, prions, protists, viruses or viroids, the action ranging from inhibition of growth to causation of death (cf.: R. B. Thurman and C. P. Gerba; “The molecular mechanisms of copper and silver ion disinfection of bacteria and viruses.” CRC Critical Reviews in Environmental Control; Vol. 18; Issue 4 (1989) ; pages 295-315). Silver, copper and zinc ions are of particular interest for a bactericidal effect, The crucial advantage of these ions compared to other bactericidal metal ions, for example Hg²⁺, is the substantial insensitivity of human metabolism thereto. The bactericidal concentration is reported to be 0.01-1 mg/l for silver and 0.1-1 mg/l for copper (Ullman's Encyclopaedia of Industrial Chemistry (5th edition), VCH 1993, Volume A 24, page 160),

Furthermore, a highly effective antiviral effect has been demonstrated for copper (cf.: S. L. Warnes, Z. R. Little and C. W. Keevil; “Human Coronavirus 2295 Remains Infectious on Common Touch Surface Materials.” mBIO 6(6):e01697-15 (2015)). Copper is capable of deactivating or killing a broad spectrum of non-enveloped (e.g. norovirus) and enveloped (e.g. influenza, SAPS-CoV-2) viruses. Both Cu (II) and Cu (I) ions are capable of damaging the cell wall of the microorganism via peroxidation. Cu(I) ions play a particular role here, since they generate, by means of the Fenton reaction, hydroxyl radicals which can destroy cellular proteins. Furthermore, when these two ions bind to the DNA of the microorganism, genetic disturbances occur, including damage to the so-called spike proteins on the surface of the virus.

This effect of heavy metals, especially silver, copper and zinc, has been employed in a diversity of fields for a long time. For instance, sensitive parts of medical devices and equipment or surfaces touched very frequently, such as handrails or door handles, are coated with copper or brass. In the case of the production of textiles, surface application (impregnation, electrodeposition, plasma coating, vapour deposition) of these metals is predominantly used, Another possibility is the introduction of zeolites or ceramics doped with metal ions. Furthermore, it is possible to produce yarns of non-metallic fibres combined with filaments of elemental silver or copper,

EP2747792 uses synthetic fibres which are mixed with copper ions before extrusion, The ions are in the form of a colloid in solid or liquid form.

In DE69633817T2, a fibre comprised of acrylonitrile with methacrylate and with or without sodium methallyl sulfonate is produced. Said fibre is cross linked with hydrazine and then hydrolysed with NaOH for the purpose of introducing carboxyl groups. After metal ions have been applied, they are in a fifth step precipitated into the fibre by means of reducing agents and heat treatment. This procedure is very complex and chemically intensive. With regard to the copper, this is not reduced to copper(I) oxide, which, as described above, is particularly suitable for an antiviral effect.

EP2371893 describes a film-forming suspension comprised of nano-scale cellulose fibres, a polyvalent metal and a volatile base.

In DE69219821T2, cellulose fibres are first treated with a metal salt solution and then with a polycarboxylic acid solution. In a fourth step, a heat treatment is carried out at 160° C., for binding of the metal ions. An antibacterial effect against Staphylococcus aureus was detected after 10 wash cycles. No antibacterial effect against Klebsiella pneumoniae or antiviral effect after 50 wash cycles was described.

CN107881763 discloses the incorporation of nano-scale copper oxide together with chitosan into a cellulose fibre. The synergy of copper oxide and chitosan results in a strong antibacterial effect. The high wash-resistance, which is not specified in further detail, is based on repeated washes with ethanol and water.

A further route is the incorporation of a natural or synthetic second component into a cellulose fibre.

DE10140772 describes Lyocell shaped articles containing algae. These exhibit a sorption capacity specifically for heavy metal ions. However, only a small amount of metal ions is bound.

DE19917614 describes the production of cellulosic shaped articles that are based on polystyrene or polyacrylate resin and have a high absorption capacity for anions and cations for use as textile ion-exchange materials. However, no details are given about the permanence of ion binding in textile use, since only single use, for example as cigarette filters and household articles, is intended.

The abovementioned EP2747792 also uses in one embodiment a proportion of superabsorbent polymers (SAP) for wound dressings which, in combination with copper ions, exhibit effective absorption of wound secretions and a bactericidal effect.

The literature (cf.: M. Turaiiia, P. Merschak, B. Redl, U. Griesser, H. Duelli and T. Bechtold; “Journal of Materials Chemistry”/B, 2015, 3, 5886-5892) describes the impregnation of textile polyester fabrics using a suspension consisting of copper(I) oxide particles, a binder and a dispersant, which fabric has antibacterial activity even after ten washes. The treated fabric was washed ten times in 250 ml bottles using a washing solution at 60° C. for 30 min.

### SUMMARY OF ADVANTAGEOUS EMBODIMENTS OF THE INVENTION

It is an object of the invention to develop a method for producing cellulosic shaped articles, the focus being a long-lasting antibacterial and antiviral effect, for use in medicine, hygiene and clothing. It is a further object of the invention to form an active ingredient depot in the fibre that substantially withstands textile processing steps and that meets the usage requirements of a textile. The latter object is associated with wash-resistance over 50 washes. Furthermore, the shaped articles produced by the method according to the invention, in particular fibres and films, shall be created such that they are suitable for producing wound dressings, sanitary products, specialty papers, and packaging and filter materials, owing to their high absorption capacity. Lastly, composites comprised of mixtures with other fibres shall be producible. In this case, an example of what shall be possible by using the fibre loaded with active ingredient is intrinsic fibre protection for nonwoven filter fabrics and nonwoven geotextile fabrics, by virtue of a fungicidal effect. A further goal of the invention is to reduce the method for producing said cellulosic shaped articles to a few steps.

### DETAILED DESCRIPTION OF ADVANTAGEOUS EMBODIMENTS OF THE INVENTION

This object is achieved by loading cellulosic shaped articles having a high absorption capacity produced by the dry-wet extrusion process, as known for example from. DE19917614 (ion-exchange fibres), with ionic active ingredients. An after-treatment step alters the active ingredient such that the active ingredient depot formed in the fibre or film is capable of releasing these active ingredients according to their equilibrium concentration over a period of at least 50 industrial standard washes such that there is antiviral and/or antibacterial activity even after a least 50 washes. The equilibrium concentration is adjustable via the ratio of the actual load to the total capacity.

Wash-resistance means that the fibre according to the invention, even after at least 50 industrial standard washes in accordance with DIN EN ISO 6330, still contains sufficient active substance for the biocidal effect to continue to be present in full. Antiviral activity is determined in accordance with the standard. ISO 18184:2019-6. Antibacterial activity is determined in accordance with the standard DIN EN ISO 20743:2013.

Absorption capacity means the absorption of metal ions in the fibre. It is determined in accordance with the standard DIN 54403:2009. According to DE19917614, absorption capacity is dependent on the nature and amount of the incorporated ion exchanger. According to the preferred embodiment of the method according to the invention, the spinning solution used for extrusion contains 1% to 200% by mass, preferably 10% to 150% by mass, based on cellulose, of the ion exchanger. The possibilty of integrating high concentrations of ion exchanger in the fibre means that it is possible to create active ingredient depots having high concentrations of metal ions in the fibre.

Active ingredient in the context of the invention are all metal salts which are water-soluble and can therefore be introduced into the fibre in an after-treatment step. This introduction occurs through interaction of the metal ion with the ionic groups of the ion exchanger in the fibre. The metal salts must additionally exhibit antiviral and/or antibacterial activity. In addition, the metal salts must be capable of being able to be converted into a sparingly water-soluble form by a further after-treatment step. This, conversion can, for example, be achieved by addition of the aqueous solution of a salt, the anion of which forms, with the metal cation of the active ingredient, a compound that is sparingly soluble in water. The conversion can, however, also be an after-treatment by means of which the oxidation state of the metal ion is changed and a sparingly soluble metal salt, metal oxide or elemental metal is formed as a result.

Metal salts which can be used as active ingredient in the invention are, for example, water-soluble silver salts such as AgNO₃, AgF, water-soluble zinc salts such as ZnSO₄, ZnI₂, ZnCl₂, ZnBr₂, Zn(ClO₃)₂, and water-soluble copper salts such as CuSO₄, CuBr₂, Cu (ClO₃)₂, CuCl₂, CuSiF₆, Cu(NO₃)₂. The conversion into water-insoluble forms is achieved by treatment with the aqueous solution of salts which form sparingly soluble salts with the metal salts of the active ingredients. These include water-soluble salts of halogens and carbonates, but also citrates, phosphates, salts of fatty acids and sulfides. Suitable salts are, for example, NaCl, NaF, NaBr, NaI, KF, KCl, KBr, KI, Na₂CO₃, NaHCO₃, Na₃PO₄, Na₂HPO₄, NaH₂PO₄, Na₂S, sodium citrate, sodium stearate.

Alternatively, active ingredients can also be converted into a different oxidation state by a redox reaction to yield water-insoluble compounds. For example, CuSO₄, which is in a first step absorbed on the fibres loaded with ion exchanger, can be reduced in an alkaline environment and in the presence of a reducing agent to copper(I) oxide, which is water-insoluble. It is also possible to reduce silver(I) salts by treatment with an aqueous solution of ammonia and a reducing agent to form elemental silver, which is likewise water-insoluble and remains in the fibre.

According to DE10315749, the concentrations of the metals, preferably silver, copper and zinc, are advantageously between 0.005 g of metal/kg of fibre to>100 g of metal/kg of fibre.

Preferred ion exchangers are polystyrene- or polyacrylate-based polymers. These can be acid-derivatised styrene-divinylbenzene or acrylic acid-divinylbenzene copolymer resins. In principle, it is also possible to use other support materials for the exchange groups, for example cellulose and cellulose derivatives.

The fibres loaded with active ingredient can be mixed with unloaded ion-exchange fibres in order to lower or control the concentration of active ingredient. In the case of the production of textile fabrics, the ion-exchange fibres loaded with active ingredient can be mixed with other natural and/or synthetic fibres, for example polyethylene, polypropylene, polyester, polyamide, polyacrylic or cellulosic fibres.

Accordingly, the invention provides a staple fibre which preferably consists of cellulose and can be shaped by a dry-wet process, such as the Lyocell process or with ionic liquids as solvents. The viscose process is also conceivable as a production process.

The ion-exchange fibre is preferably loaded by an immersion process in which the fibre is soaked with a salt solution containing, for example, silver, copper or zinc ions. The fibre is then washed with water and spun down multiple times. After soaking in a finishing bath, the fibre is once again spun down and dried.

According to wash experiments, this fibre exhibits only low wash-resistance. A loss of approx. 90% of the metal can be observed after just 10 washes. Therefore, according to the preferred embodiment of the method according to the invention, the loading procedure was extended by a further step: fixing the metal ion in the fibre. What was found was that, surprisingly, the treatment of the already-loaded fibre with a second salt solution containing chloride or carbonate ions leads to the formation of a compound which is sparingly soluble in water and which is bound more firmly in the fibre and consequently gives the fibre a distinctly higher wash-resistance. Silver chloride and copper and zinc carbonates are practically insoluble in water. For instance, distinct indications of the presence of, for example, AgCl crystallites in the fibre were detected by means of wide-angle X-ray scattering (WAXS). FIG. 1 shows the WAXS spectrum of a fibre loaded with silver ions and having had additional NaCl fixation in comparison with the spectrum of the pure AgCl compound.

The distinctly higher wash-resistance was demonstrated by means of domestic washing at 40° C. with a customary heavy-duty laundry detergent over 50 wash cycles. FIG. 2 shows, as an example, the measurement of the copper contents of the fibre with and without Na₂CO₃ fixation.

In a further embodiment, the fixation takes place with a simultaneous change in the oxidation number of the copper. It was found that, surprisingly, a fibre loaded with divalent copper ions can be converted into a fibre containing integrated monovalent copper oxide by means of a basic glucose solution.

This copper(I) oxide (Cu₂O) is likewise firmly bound in the fibre. Its crystal structure was confirmed by WAXS measurements (see FIG. 3). Besides the higher wash-resistance, an effective antiviral effect is achieved, as described above. Since copper(I) compounds are unstable in air, they gradually oxidize back to divalent copper. Therefore, it is not expedient to, for example, load a fibre with Cu₂O particles. The intrinsic introduction of the Cu₂O into the cellulose fibre therefore provides protection from oxidation. This Cu₂O depot that is formed ensures a long-lasting (permanent) antiviral effect.

Despite the incorporation of the metal in a fixed form as carbonate, chloride or oxide, there are still sufficient free ions present in the cellulose structure according to surface equilibrium reactions, for example metal carbonate ⇔metal ion+carbonate ion. Owing to the effect of the cellulose as a hydrophilic network-forming polymer having a residual moisture content of up to 15%, transport of the metal ion from the interior of the fibre to the surface is always ensured. Therefore, the metal ions in the interior of the fibre and at the surface of the fibre are fully available to exhibit their antibacterial or antiviral effect on microorganisms. Even during relatively long periods of use of the fibre, surface levels are always replenished from the internal active ingredient depot. The equilibrium concentration of the metal ions is sufficient for the biologically effective range. Both the antibacterial (Ag, Cu, Zn) and the antiviral effect (Cu) were detected. Even after 50 wash cycles, only a loss of the antibacterial or antiviral effect was registered.

### BRIEF DESCRIPTION OF THE DRAWINGS

FIG. 1 shows a spectrum generated by wide-angle X-ray scattering (WAXS) of a fibre loaded with silver ions in which the silver ions were subsequently fixed with sodium chloride (top) and a spectrum of pure silver chloride for comparison (bottom).

FIG. 2 shows the proportion of copper in an SAP fibre loaded with copper sulfate, with or without fixation by a sodium carbonate solution, according to the number of washes carried out.

FIG. 3 shows the WAXS spectrum of a fibre which has been loaded with divalent copper ions and then treated with a basic glucose solution (top) and a spectrum of pure copper (I) oxide for comparison (bottom).

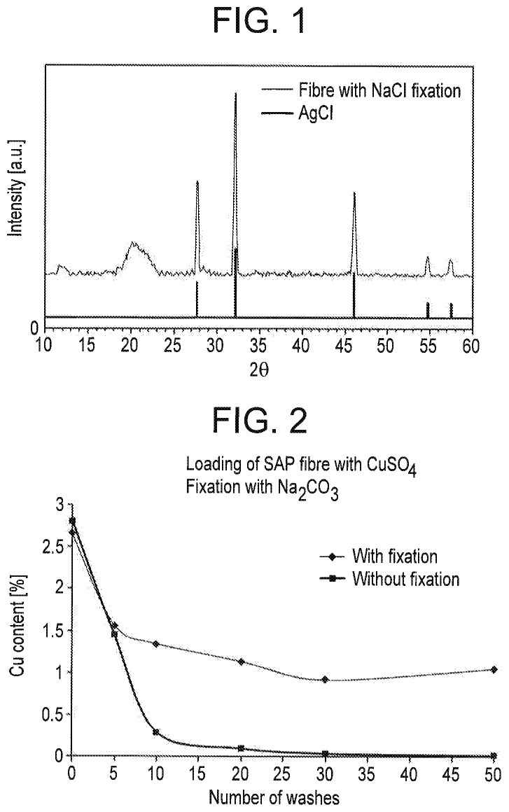

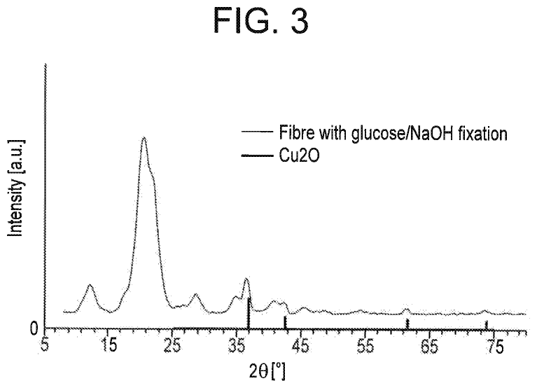

### EXAMPLES

The following examples serve to further illustrate the method according to the invention.

Test methods for determination of element contents and for assessment of antibacterial and antiviral activity of cellulosic fibres:

The antibacterial effect of the fibres was determined in accordance with test standard DIN EN ISO 20743 “Textiles—Determination of antibacterial activity of textile products” by application of a defined number of bacteria in dilute nutrient solution to the fibres and incubation thereof at 37° C. for 24 h. The bacteria were then detached by means of shaking and the number of surviving bacteria that remained was determined by means of a plating method. From the logarithms of the bacterial cell counts obtained for a sample without an antibacterial finish (control material) and for the antibacterial fibres, the difference was calculated, said difference representing a measure of antibacterial activity, with a log 2 reduction meaning good antibacterial activity and a log 3 reduction meaning very good activity.

Antiviral activity was determined in accordance with test standard ISO 18184 “Textiles—Determination of antiviral activity of textile products”. To this end, the enveloped bacteriophage phi6 was used as surrogate virus for the human enveloped viruses influenza A or SARS-CoV-2 and applied in a defined number to the fibres and incubated at 25° C. for 2 h. The phages were then detached by means of shaking and the number of surviving phages that remained was determined by means of a plaque titre assay. From the logarithms of the plaque titres obtained for a sample without an antiviral finish (control material) and for the antiviral fibres, the difference was calculated, said difference representing a measure of antiviral activity, with a log 2 reduction meaning low antiviral activity and a log 3 reduction meaning full antiviral activity.

Copper, silver and. zinc contents were determined by ICP-OES in accordance with DIN EN ISO 11885 after microwave pressure digestion.

The WAXS measurements were carried out using a BRUKER D8 Advance series instrument equipped with a position-sensitive row detector, in symmetric transmission. Measurement was carried out by using Cu K_{α} radiation of wavelength λ=0.1542 nm (doublet) with a tube voltage of 40 kV and 40 mA anode current and the K_{β} portion filtered out. The test specimens prepared were tablets of uniform density and a thickness of 2 mm.

Chemicals used:
Sodium polyacrylate, cross-linked (CAS: 9033-79-8, PRODUCT T 5066 F, from Evonik)
Copper(II) sulfate pentahydrate (CAS: 7758-99-8, purity ≥98%, from, for example, VWR)
Sodium carbonate (CAS: 497-19-8, purity ≥98%, from, for example, VWR)
Glucose monohydrate (CAS: 14431-43-7, purity ≥98%, from, for example, VWR)
Sodium chloride (CAS number: 7647-14-5, purity ≥98%, from, for example, VWR)
AFILAN® RA (octadecanamide, CAS number: 10220-90-3, from Archroma)

### Example 1

The ion-exchange fibres, produced according to DE19917614 and containing a proportion of 15% sodium polyacrylate, are treated with a copper sulfate solution. To this end, 15 kg of the ion-exchange fibre are washed with deionized water and then loaded with a 0.15 M aqueous copper sulfate solution. After a residence time of 20 min in this solution with intensive stirring, the fibres are spun down and centrifuged. In a second treatment bath, the fibres are finished using a customary softener, for example AFILAN® RA, The fibres have a linear density of 6.7 dtex, an elongation of 10% and a breaking strength of 21 cN/tex. The copper concentration is 28 000 mg/kg copper. After 50 wash cycles, the fibres still contained 200 mg/kg copper. Measurement of the antibacterial effect versus Staphylococcus aureus showed a reduction of log 5.8 and, after 50 wash cycles, log 5.3; versus Klebsiella pneumoniae, there was a reduction of log 5.8 and, after 50 wash cycles, log 5.5. For both bacterial species, this signifies strong antibacterial activity that is still maintained even after 50 wash cycles. Measurement of the antiviral effect against Pseudomonas sp. DSM 21482 revealed a log 3.0 reduction, which corresponds to a strong antiviral effect.

### Example 2

Ion-exchange fibres produced according to Example 1 are, following copper loading, additionally placed in a second immersion bath containing a 10 g/l sodium carbonate solution and stirred therein for 20 minutes. The fibres are then spun down and centrifuged. In a third treatment bath, the fibres are finished according to Example 1. The copper concentration is 26 500 mg/kg copper. After 50 wash cycles, the fibres still contained 10 400 mg/kg copper. Compared to the non-fixed fibre from Example 1, this corresponds to an increase in recovery after 50 washes from approx. 0.7 to approx. 39%.

### Example 3

Ion-exchange fibres produced according to Example 1 are, following copper loading, additionally placed in a second immersion bath containing a solution containing 10 g/l glucose and 5 g/l NaOH and stirred therein for 20 minutes. The fibres are then washed, spun down and centrifuged multiple times until a neutral reaction is achieved. In a third treatment bath, the fibres are finished according to Example 1. The copper concentration is 7890 mg/kg copper, After 50 wash cycles, the fibres still contained 321 mg/kg copper. The antibacterial effect after 50 wash cycles versus Staphylococcus aureus showed a reduction of log 4.7, and versus Klebsiella pneumoniae a reduction of log 4.4, The antiviral effect against Pseudomonas sp. DSM 21482 showed a log 4.5 reduction and, after 50 washes, still a reduction of log 4.1. This corresponds to strong antiviral activity even after 50 washes.

### Example 4

Ion-exchange fibres produced according to Example 1 are processed in a 6% mixture, with pure Lyocell fibres into a needle-punched nonwoven. The antibacterial effect versus Staphylococcus aureus was determined as a log 5.6 reduction; after 20 wash cycles, the reduction was still log 5.3. Versus Klebsiella pneumoniae, there was a log 5.9 reduction and, after 20 wash cycles, a log 4.4 reduction.

### Example 5

Ion-exchange fibres produced according to Example 1, but treated with 0.15 M aqueous silver nitrate solution instead of copper sulfate. The fibres have a linear density of 6.7 dtex, an elongation of 11% and a breaking strength of 23 cN/tex. The silver concentration is 51 200 mg/kg silver. After 50 wash cycles, the fibres still contained 2150 mg/kg silver.

### Example 6

Ion-exchange fibres produced according to Example 5 are, following silver loading, additionally placed in a second immersion bath containing a 10 g/l sodium chloride solution and stirred therein for 20 minutes, The fibres are then spun down, centrifuged and finished according to Example 2. The silver concentration is 48 300 mg/kg silver. After 50 wash cycles, the fibres still contained 14 500 mg/kg silver. Compared to the non-fixed fibre from Example 3, this corresponds to an increase in recovery after 50 washes from approx. 4% to approx. 30%, Measurement of the antibacterial effect versus Staphylococcus aureus showed a reduction of log 6.0 and, after 50 wash cycles, of log 5.8; versus Klebsiella pneumoniae, there was a reduction of log 6.0 and, after 50 wash cycles, of log 5.6. For both bacterial species, this signifies strong antibacterial activity which is still maintained even after 50 wash cycles.

1. A method for producing a cellulosic fibre loaded with a biologically active substance, wherein said method comprises of the steps:
(a) producing a cellulosic fibre loaded with ion exchanger;
(b) after-treating the fibre in step (a) with an aqueous solution of a metal salt which exhibits antibacterial activity and/or antiviral activity; and
(c) after-treating the fibre in step (b) with an aqueous fixing solution to convert the metal salt into a water-insoluble form.

2. The method according to claim 1, wherein said metal salt comprises water-soluble silver, copper or zinc salts.

3. The method according to claim 2, wherein said metal salt comprises CuSO₄, AgNO₃ and ZnSO₄.

4. The method according to claim 1, wherein said aqueous fixing solution comprises salts having anions selected from F₋, Cl₋, Br⁻, I⁻, ClO₃⁻, ClO₄⁻, CO₃²⁻, HCO₃⁻, PO₄³⁻, HPO₄², H₂PO₄⁻, S²⁻, citrate, and salts of fatty acids.

5. The method according to claim 1, wherein said aqueous fixing solution comprises a base a reducing agent.

6. The method according to claim 5, wherein said base is NaOH or ammonia and said reducing agent is an aldehyde.

7. A cellulosic fibre produced according to the method of claim 1.

8. The cellulosic fibre according to claim 7, further comprising textile fibre, wherein said textile fibre is mixed with said cellulosic fibre to produce textile fabric(s).

9. The cellulosic fibre according to claim 8, wherein said textile fibre comprises polyethylene, polypropylene, polyester, polyamide, polyacrylic or cellulosic fibre.

10. The cellulosic fibre according to claim 7, wherein said cellulosic fibre is used for producing wound dressings, sanitary products, specialty papers, packaging and filter materials.

11. The cellulosic fibre according to claim 7, wherein said cellulosic fibre is used as intrinsic fibre protection for nonwoven filter fabrics and nonwoven geotextile fabrics.
